(19) United States
(12) Patent Application Publication
(10) Pub. No.: US 2025/0073456 A1
(43) Pub. Date: Mar. 6, 2025

(54) Cuff For Providing Vagus Nerve Stimulation

(71) Applicant: United States Government As Represented By The Department Of Veterans Affairs, Washington, DC (US)

(72) Inventor: Matthew Schiefer, Newberry, FL (US)

(21) Appl. No.: 18/826,930

(22) Filed: Sep. 6, 2024

Related U.S. Application Data
(60) Provisional application No. 63/580,881, filed on Sep. 6, 2023.

(51) Int. Cl. A61N 1/05; A61N 1/36

(52) U.S. Cl. CPC A61N 1/0556; A61N 1/36053

(57) ABSTRACT

A cuff for providing vagus nerve stimulation to a subject having an esophagus is disclosed. The cuff includes a helical body that is configured to wrap around at least a portion of the esophagus of the subject, the helical body having an inner surface, the inner surface having a length and a width. The inner surface defines an interior space that is configured to receive the esophagus. A first electrode is coupled to the helical body. At least one additional electrode is coupled to the helical body. The at least one additional electrode is configured to provide a polarity opposite of the first electrode.

### CROSS-REFERENCE TO RELATED APPLICATION

This application claims priority to and the benefit of the filing date of U.S. Provisional Patent Application No. 63/580,881, filed Sep. 6, 2023, the entirety of which is hereby incorporated by reference herein.

### FIELD

This application relates to apparatuses and methods for providing vagus nerve stimulation, for example, to inhibit appetite of a subject.

### BACKGROUND

Obesity, defined as a body mass index (BMI) ≥30 kg/m², is prevalent in the US. Beyond the healthcare crisis of obesity, the economic costs of obesity are also staggering. In a worsening trend, 31 states have obesity rates ≥30% and none are below 20%. From 2008 to 2018, the U.S. saw a year-over-year increase in these high obesity rates of 23%. More than 30% of U.S. adults are obese and >70% of Veteran service personnel are estimated to be overweight or obese. In the U.S., nearly 10% of all deaths are obesity-related. The Surgeon General noted that obesity is a top priority that poses a significant threat to military readiness and national security if left unaddressed. According to a 2017 Pentagon report, more than 1 in 5 Americans aged 17-24 are ineligible to serve in the U.S. Military due to obesity. Obesity-related expenses are nearly 20% of annual U.S. healthcare expenditures. Recognizing this, the Federal government devotes more than $1B annually to obesity research.

Diet and exercise can reduce excess body weight (EBW), but 30%-60% of dieters relapse. Gastric banding has the lowest mortality rate (0.1%) as a treatment for obesity, but has the highest long-term failure rate (>50%). Roux-en-Y gastric bypass (RYGB) and sleeve gastrectomy have lower failure rates but higher mortality rates. Intragastric balloons are less invasive but also less effective than RYGB. Approximately 230,000 Americans undergo bariatric surgery annually.

### SUMMARY

Disclosed herein, in one aspect, is a cuff for providing vagus nerve stimulation to a subject having an esophagus. The cuff includes a helical body that is configured to wrap around at least a portion of the esophagus of the subject, the helical body having an inner surface, the inner surface having a length and a width. The inner surface defines an interior space that is configured to receive the esophagus. A first electrode is coupled to the helical body. At least one additional electrode is coupled to the helical body. The at least one additional electrode is configured to provide a polarity opposite of the first electrode.

Methods of using the disclosed cuff are also disclosed.

### BRIEF DESCRIPTION OF THE DRAWINGS

The accompanying drawings, which are incorporated in and constitute a part of this specification, illustrate several embodiments of the disclosed apparatus, system, and method and together with the description, serve to explain the principles of the disclosed apparatus, system, and method.

FIGS. 1A and 1B show fMRI images of rat brains on a standard diet and a high glucose diet, respectively. During sVNS, rats on a standard diet had a significant increase in activity in the satiety regions (ventromedial hypo-thalamus (VMH), red outline; dorsal medial nucleus (DMN), blue outline). The VMH plays a major role in terminating hunger and the DMN is involved with feeding activity and body weight regulation. Conversely, rats on an HG diet exhibited a significant increase in activation in food-based reward regions (lateral hypothalamus (LH), green outline; ventral tegmental area (VTA), not visible). The LH is associated with increased food craving and suppression of distension-driven inhibitory feedback. Both the LH and VTA are part of the feeding reward system that can drive overconsumption of highly palatable food.

FIG. 2 shows a 3D computer model of the helical cuff as disclosed herein comprising staggered cathodes flanked by two anode ribbons. Computer simulations were run to optimize the width of the silicone cuff wall (SW), the pitch of the silicone (SP), the width of each cathode (CW), the length of each cathode (CL), the width of both anodes (AW), and the edge-to-edge spacing between staggered cathodes (CC) and the cathode and nearest anode (CA).

### DETAILED DESCRIPTION

The disclosed system and method may be understood more readily by reference to the following detailed description of particular embodiments and the examples included therein and to the Figures and their previous and following description.

It is to be understood that the terminology used herein is for the purpose of describing particular embodiments only and is not intended to limit the scope of the present invention which will be limited only by the appended claims.

It must be noted that as used herein and in the appended claims, the singular forms “a,” “an,” and “the” can include both single and plural references unless the context clearly dictates otherwise. Thus, for example, reference to “an electrode” includes aspects in which only a single spring is provided, as well as aspects in which a plurality of such electrodes are provided.

“Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Optionally, in some aspects, when values or characteristics are approximated by use of the antecedents “about,” “substantially,” or “generally,” it is contemplated that values within up to 15%, up to 10%, up to 5%, or up to 1% (above or below) of the particularly stated value or characteristic can be included within the scope of those aspects.

Unless defined otherwise, all technical and scientific terms used herein have the same meanings as commonly understood by one of skill in the art to which the disclosed apparatus, system, and method belong. Although any apparatus, systems, and methods and materials similar or equivalent to those described herein can be used in the practice or testing of the present apparatus, system, and method, the particularly useful methods, devices, systems, and materials are as described.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step. As used in the specification and in the claims, the terms “comprises” and “comprising” can include the aspects “consists of,” “consisting of,” “consists essentially of,” and/or “consisting essentially of.”

The following describe various aspects of certain illustrated embodiments. It should be understood that the embodiments are provided merely as examples, and aspects of each illustrated embodiment can be incorporated into the other embodiment. Further, each illustrated embodiment can be modified further to provide additional embodiments consistent with the present disclosure.

### Overview

The vagus nerves convey the degree of stomach fullness to the brain via afferent axons. Nearly all abdominal vagus fibers are unmyelinated sensory afferents, including stretch-sensitive mechanoreceptors in the walls of the stomach and intestines. As the stomach stretches during food intake, the discharge rate of these mechanoreceptors increases. Tension-sensitive mechanoreceptors are a strong influencer of afferent discharge and play a critical role in establishing satiety and reductions in the mechanosensitivity of these receptors contribute to hyperphagia (excessive cating). For example, mice lacking the transient receptor potential vanilloid 1 (TRPV1) channel are less sensitive to stomach stretch and exhibit increased food intake. Stomach volume has the strongest influence on afferent discharge and inhibition of food intake, more so than stomach contents. Thus, bariatric surgery and gastric balloons are successful because they decrease stomach volume, which increases stomach wall stretch and vagus activity as food is consumed, thereby eliciting fullness while consuming less volume and, presumably, fewer calories.

The FDA approved cervical vagus nerve stimulation (VNS) to treat intractable epilepsy and chronic depression. There are ongoing investigations into the use of VNS to treat or modulate myriad conditions including migraine headache, cardiac arrhythmia, anxiety, Alzheimer's, fibromyalgia, tinnitus, post-traumatic stress disorder, and inflammation. Reduction in excess body weight (EBW) has been noted as a side effect of VNS. Subsequently, researchers showed that VNS promotes reduced food intake, causes weight loss and reduces cravings and appetite in animals. Retrospective and case studies show similar evidence in humans. VNS can also decrease acid reflux and gastric motility; increase satiety (being “satisfied” or “full”) and gastric volume; improve glucose tolerance; disrupt peristalsis; and induce tachygastria. It is contemplated that VNS can decrease EBW by increasing vagus afferent activity, mimicking stomach distension and promoting satiety. An alternative method, high frequency stimulation to block the vagus nerve (VBLOC), arose because studies reported that abdominal vagotomy (nerve transection) elicited weight loss. VBLOC has induced weight loss that reached significance by 18 months, but long-term follow-up suggests limited efficacy. VBLOC may work by blocking aberrant signals that promote hyperphagia. Alternatively, a strong, transient onset response may increase expression of receptors sensitive to leptin and CCK, thereby hypersensitizing the nerve to distension. Computer simulations suggest VBLOC both blocks and activates different axon populations in the nerve. The novel interface disclosed herein can deliver VNS or VBLOC.

Non-invasive imaging can help better understand the effects of VNS on the brain. Blood oxygen level dependent (BOLD) functional magnetic resonance imagining (fMRI) can reveal patterns of activation and inhibition within and between areas associated with satiety and food-based reward (i.e., “connectomics”). Understanding how healthy animals versus obese animals perceive VNS and changes in stimulus parameters can open the door to fast stimulus optimization that is not feasible when using weight loss as a metric because healthy weight loss occurs over a very long time period. Due to the range of successful VNS parameters and conflicting findings, the American Society for Metabolic and Bariatric Surgery called for more studies to understand VNS. Better understanding the effects of stimulus parameters both peripherally and centrally and how those effects contribute to VNS efficacy can permit widespread clinical deployment.

Despite its potential, VNS for obesity has been preclinical. Optimal stimulus parameters can be determined. Optimization is challenging given the delay required from a change in stimulus parameter to a change in food consumption habits and/or weight loss. Reported VNS parameters vary significantly. Wide-ranging stimulus parameters have been tested: frequencies from 0.05 to 30 Hz; pulse widths (PW) from 0.025 to 500 ms; pulse amplitudes (PA) from 0.25 to 15 mA; trains from 14 to 43,200 s; studies from 10 to 100 days. In most studies, VNS resulted in weight loss, decreased food intake, and/or decreased adiposity, but noted that the selected stimulus parameters had no clear justification. Further, VNS pulses typically are static with an unchanging PW and PA, thereby establishing a static population of activated axons. This is not natural. Recently the use of dynamic pulses demonstrated that more sophisticated waveforms can alter brain activity and consumption of specific food types. Data indicates that dynamic stimuli can establish a healthy body weight in a rat maintained on a high fat diet that would otherwise be obese.

Beyond stimulus parameters, there is also a choice of location. VNS can be unilateral or bilateral. It can be applied at the cervical level (cVNS), the subdiaphragmatic level (sVNS), or via gastric wall electrical stimulation (GES). Each location has advantages and disadvantages. cVNS can recruit all vagus visceral axons in the cervical vagus nerves (cVN) but elicits side effects that can alter cardiac output and respiration. Targeting the right cVN is dangerous and typically avoided. Unintended recruitment of myelinated motor fibers that form the recurrent laryngeal nerve is difficult to avoid, which can interfere with swallowing. GES mostly eliminates the risk of off-target side effects, but the recruited axon population is limited to the fibers passing near or under the electrodes on the stomach wall. Because the subdiaphragmatic vagus nerves (sVN) contain solely vagus visceral axons the sVN are an ideal target. Bilateral sVNS maximizes target axon recruitment while minimizing off-target effects. Thus, sVNS has the potential to serve as a fully reversible, less-invasive option to bariatric surgery. While sVNS can be optimized in a rat model, technological limitations of conventional technology has presented challenges.

Bilateral VNS is more effective than unilateral VNS, presumably because bilateral stimulation recruits a larger axon population and the interpreted intensity of stomach stretch (and resulting perceived fullness) is proportional to the size of the recruited axon population, similar to our findings in other sensory systems. However, bilateral VNS has not been the preferred method. As noted, bilateral cVNS carries significant off-target risks. Typically bilateral sVNS is not attempted in rats or mice because the sVN are small and adhered to the esophagus, one on the ventral side and one on the dorsal side, though possibly offset laterally. Surgically freeing the posterior sVN from the esophagus is very challenging, requiring a 180° rotation of the stomach and esophagus to visualize and surgically free the very fragile nerve. For both sVN, surgically freeing the nerve from the esophagus carries a significant risk of permanent nerve damage. Further, the inherently small nerve cuff size means small electrode surface area, but the unmyelinated target axons require a larger stimulus charge that can drive a small electrode outside the water window and safe operating range.

It was discovered that a cuff positioned around the entire distal esophagus enveloping both sVN produces bilateral sVNS while minimizing nerve manipulation. Such a cuff is useful in non-survival experiments, but it introduces a significant risk in survival experiments: esophageal stenosis caused by a fibrotic encapsulation ring. In extreme cases, this prevents passage of food into the stomach and requires euthanasia. A loose cuff reduces the likelihood of stenosis, but also the effectiveness of sVNS. Thus, the cuff must not be too tight nor too loose at the time of implantation.

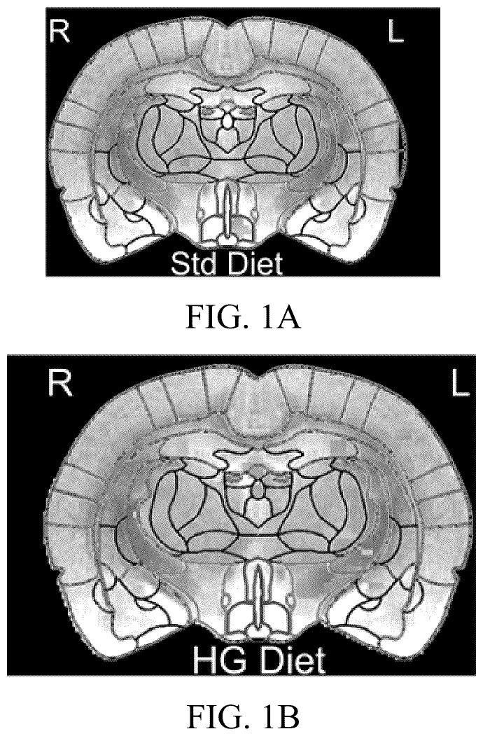

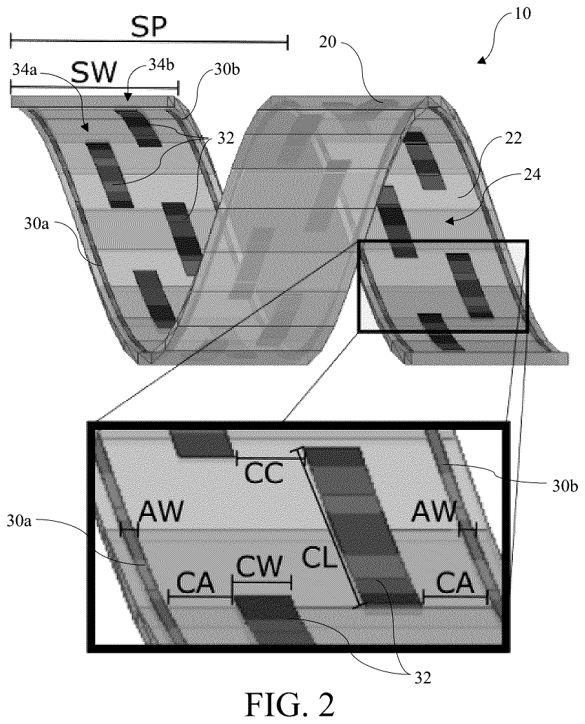

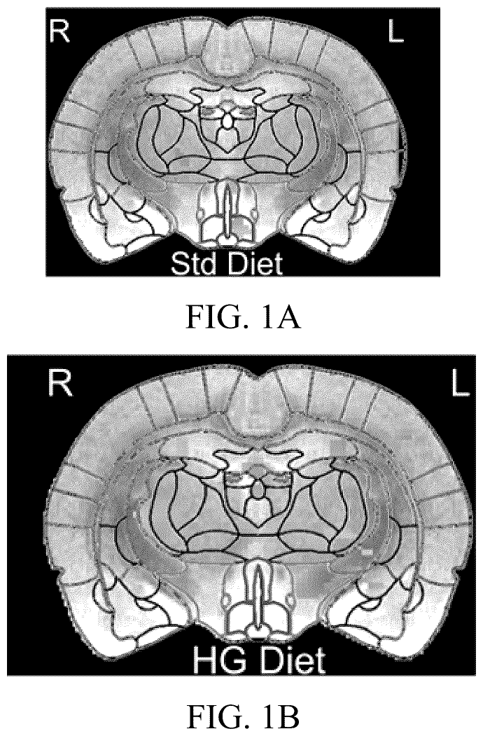

fMRI can be used to infer perception and monitor neuroplasticity in animals. While using a 7 T MRI, bilateral sVNS was applied through a standard (non-helical) circumesophageal cuff to rats aged 80 weeks. Data showed that rats on a standard (Std) diet showed significant activation in satiety regions while rats on a high glucose (HG) diet showed activation in reward regions, as illustrated in FIGS. 1A-1B. These fMRI data are promising, but the resolution from the 7 T scanner is suboptimal because voxels straddle important borders, particularly in smaller sub-regions of the hypothalamus. Additionally, the signal-to-noise ratio resulted in a larger than desirable variance in the datasets. While a longitudinal study would allow sufficient time to search parameter space and optimize the bilateral sVNS paradigm, this is not possible with the traditional cuff due to the risk of esophageal stenosis. To overcome the risk of stenosis, a novel helical cuff was designed that, when encapsulated, does not form a ring.

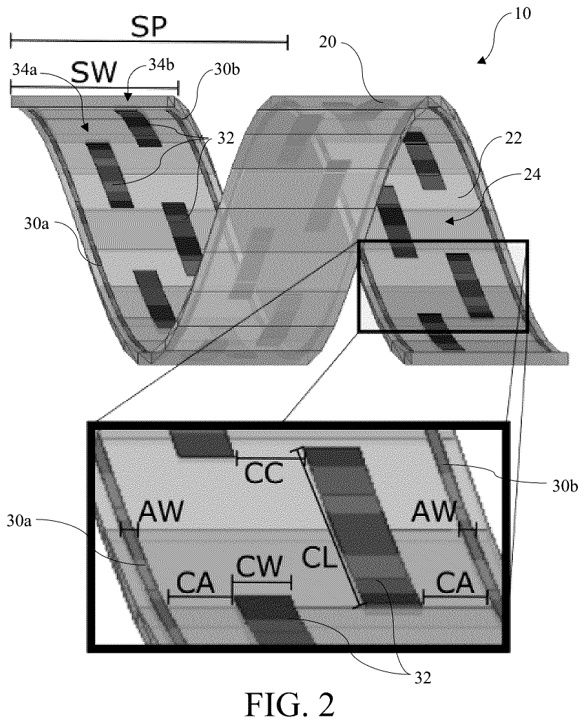

To optimize the sVNS cuff, aver 500 3D finite element models (FEMs) were constructed via Ansys Maxwell to simulate a variety of helical cuff designs in which several dimensions, as indicated in FIG. 2 were systematically varied. The cuff was modeled around an esophagus that had a ventral and a dorsal sVN, the locations of which were systematically varied relative to each other and to the orientation of the cuff. The sVN axons' responses to stimulation were simulated in Matlab. Simulations sought to maximize the percentage of axons within both sVN that were activated while minimizing the leakage of current beyond the nerves. The theoretically optimal outcome was discussed with two leading cuff manufacturers to ensure that fabrication was feasible. A cuff with distributed cathodes, as illustrated in FIG. 2, was found to be advantageous over on having a single-ribbon-like cathode similar to the anodes because the required stimulating current was significantly lower with a cuff with distributed cathodes. Further, a cuff with distributed cathodes can provide selective unilateral sVNS.

### Helical Cuff

Disclosed herein, in various aspects, and with reference to FIG. 2, is a cuff 10 for providing vagus nerve stimulation to the esophagus of a subject. The cuff 10 comprises a helical body 20 that is configured to wrap around at least a portion of the esophagus of the subject. The helical body 20 can have an inner surface 22, the inner surface having a length and a width. The inner surface 22 can define an interior space 24 that is configured to receive the esophagus. The cuff 10 can further comprise a first electrode 30a coupled to the helical body and at least one additional electrode 32 coupled to the helical body that is configured to provide a polarity opposite of the first electrode. For example, the first electrode 30a can be an anode, and the at least one additional electrode 32 can be a cathode. It is contemplated, however, that the polarities can be reversed without departing from the scope of this disclosure.

In some optional aspects, the cuff 10 can comprise a plurality of electrodes 32 (e.g., cathodes) that are configured to provide a polarity opposite of the first electrode 30a (e.g., an anode).

In some optional aspects, the first electrode 30a can be helical. In further optional aspects, the electrode(s) 32 can be helical. In other aspects, the first electrode 30a and the electrode(s) 32 can have any suitable shape. For example, they can be rectangular, circular, oval or irregular.

In some aspects, the first electrode 30a and electrode(s) 32 can comprise metal. The first electrode 30a and electrode(s) 32 can be coupled to the inner surface 22 of the helical body 20. Optionally, in these aspects, the electrodes (e.g., the first electrode 30a and electrode(s) 32) can be coupled to the helical body 20 by integral formation. For example, in some aspects, the cuff 10 can be embodied as a flex circuit.

Optionally, the cuff 10 can include only a first electrode 30a and no additional electrodes that provide the same polarity. In other aspects, the cuff 10 can comprise a second electrode 30b that is configured to provide the same polarity as the first electrode 20a. For example, the cuff 20 can comprise two anodes. In some aspects, the first and second electrodes 30a,b can be spaced along the width of the inner surface 22 of the helical body 20. Optionally, in these aspects, the electrode(s) 32 (providing the polarity opposite the first and second electrodes 30a,b) can be positioned between the first and second electrodes 30a,b. In some aspects, the second electrode 30b can be helical.

In various aspects, cuff 10 can comprise a plurality of electrodes 32 spaced along the length of the inner surface of the helical body 20. For example, the plurality of electrodes 32 can be arranged in a helical fashion. Optionally, in these aspects, the plurality of electrodes 32 spaced along the length of the inner surface of the helical body can comprise a first row 34a of electrodes 32 and a second row 34b of electrodes 32. The first row 34a can be spaced from the second row 34b along the width of the inner surface 22 of the helical body 20. In some aspects, the electrodes of each row can extend longitudinally past the electrodes of the other row to provide a longitudinal overlap. That is, a line extending across the width of the helical body 20 from one longitudinal end of an electrode 32 in the first row 34a can intersect a portion of an electrode 32 in the second row. The length of the body 20 can extend along the inner surface 22, along a helical path.

Referring to FIG. 2, for an exemplary cuff 10 that is configured for use with a rat, the helical body 20 can have a width of about 3.5 mm. The electrodes 32 can have a width from about 0.5 mm to about 0.75 mm. The electrodes 32 can have a length from about 1.34 mm to about 1.87 mm. In some aspects, electrodes 32 in the first and second rows 34a,b can have longitudinal overlaps from 0.05 mm to 1.0 mm. It should be understood that these are merely exemplary dimensions for certain embodiments, but further dimensions are contemplated. Further, the dimensions can be adjusted for a given subject. For example, it is contemplated that a cuff 20 for human subjects can advantageously have larger spacing than the spacing for rat subjects. Further, a cuff for human subjects can advantageously have larger electrodes than those for rat subjects.

In some aspects, the first electrode 30a can extend a majority of the length of the inner surface of the helical body 20. In further aspects, the second electrode 30b can extend a majority of the length of the inner surface 22 of the helical body 20.

In some optional aspects, the helical body 20 can have a consistent pitch. In other aspects, the pitch of the helical body 20 can vary along the length of the inner surface. In some aspects, the helical body can have a consistent radius. That is, the helical body 20 can follow the path of a circular helix. Optionally, the helical body 20 can include at least one full revolution. For example, the helical body 20 include at least 1.5 revolutions, at least 2 revolutions, or more (e.g. up to 20, up to 15, up to 10, or up to 5). In other aspects, the helical body 20 can have less than a single revolution. For example, the helical body 20 can have from about ½ revolution to about 1 revolution, or about ¾ of a revolution.

The helical body 20 can be configured to resiliently expand to increase a diameter of the interior space 24 defined by the inner surface 22 of the helical body to permit swallowing of the subject. Accordingly, in exemplary aspects, the helical body 20 can comprise silicone or other biocompatible, flexible material.

### Alternative Cuff Configurations

Although a cuff having a helical body is disclosed, other structures that partially or completely surround the esophagus of the subject are contemplated. For example, an exemplary cuff can encircle some or all of the esophagus, the exemplary cuff being configured to expand to increase an inner operative radius as the esophagus expands to permit the subject to swallow. For example, the cuff can comprise a split ring that is configured to encircle the esophagus of the subject. For example, the split ring can comprise a cylindrical inner surface and a pair of axially extending edges. Optionally, the axially extending edges can be parallel to a central axis of the split ring and spaced apart in faced relation. The split ring can be configured to resiliently expand to modify the spacing between the pair of axially extending edges. In some aspects, the cuff can comprise a material that is sufficiently resilient to permit expansion of the esophagus during swallowing.

### Method of Using Cuff

A method of providing vagus nerve stimulation to a subject having an esophagus and a vagus nerve can comprise positioning the cuff 10 around the esophagus of the subject. The cuff 10 can be used to apply stimulation to the vagus nerve of the subject.

In some aspects, the stimulation can be unilateral stimulation. In other aspects, the stimulation can be bilateral stimulation.

The subject can be a human subject or an animal subject (e.g., a rat, a mouse, or another laboratory mammal).

In some aspects, the stimulation can be applied to a ventral subdiaphragmatic vagus nerve (or anterior vagus nerve) of the subject. In some aspects, the stimulation can be applied to a dorsal subdiaphragmatic vagus nerve (or posterior vagus nerve) of the subject.

In some aspects, the cuff can apply a stimulation routine that is configured to inhibit appetite of the subject. In various aspects, the stimulation routine can include a current from 0 to 3 mA. For example, the current can range from 2 mA to 3 mA. The stimulation routine can include a stimulation duration from 0-1000 milliseconds (ms), or from about 500 ms to about 1000 ms, or about 800 ms. In some aspects, the stimulation routine can have a frequency from about 15 Hz to 50 Hz, or about 30 Hz. It is further contemplated that a dynamic stimulation routine can be preferred over steady-state stimulation routine.

It is contemplated that the stimulation routine can be optimized for a given subject or a given group of subjects. Accordingly, the stimulation routine can vary from the ranges of parameters disclosed herein. For example, the stimulation routine can be varied based on distance from the electrode to the nerve. The larger the distance, the higher the stimulus current and/or pulse width that can be used. The distance from the electrode to the nerve can be influenced by anatomy relative to the nominal size of the cuff. For example, if the cuff is slightly larger than the esophagus, then the distance from the electrode in the cuff to the nerve on the esophagus can increase. The stimulation routine can further be varied based on formation of encapsulation tissue due to a foreign body response. The severity of the foreign body response can affect how thick the encapsulation tissue is. Thicker encapsulation tissue on the inner surface of the cuff can push the electrode farther from the nerve.

### EXEMPLARY EMBODIMENTS

### Research Design and Methods

An exemplary study can use 34 Sprague Dawley (SD) rats assigned to four Groups and three Aims (Table 1). There can be an equal number of males and females in each Group. Aim 1 focuses on efficacy. It maps the relationship between sVNS parameters and axon recruitment recorded in the cVN. Axon recruitment by the novel cuff can be 1) compared to that of a traditional cuff as a gold standard; and 2) compared at the time of surgery and 20 weeks after surgery to assess stability. Aim 2 can focus on safety and efficacy when applying bilateral sVNS through the novel cuff in an 11.1 T magnetic field. It can determine the amount of tissue heating during MRI scans. It can also map the relationship between sVNS parameters and brain activation/inhibition to determine if diet alters response. Aim 3 can focus on safety. It can determine if the novel cuff has reduced or eliminated the risk of esophageal stenosis and if the cuff damaged sVN axons. The novel cuff can be deemed fully successful if it 1) is easy to implant; 2) bilaterally recruits most of the target axons innervating the stomach without apparent off-target effects; 3) exhibits no damage to the sVN; 4) exhibits no esophageal stenosis; 5) exhibits no heating during fMRI; and 6) does not introduce artefacts into the fMRI data.

TABLE 1
Animals Used In This Example

| Group | N | Diet | Cuff | Surgery | Aim(s) |
|---|---|---|---|---|---|
| “A” | 10 | Std | Traditional | Non-Survival | 1, 3 |
| “B” | 10 | Std | Novel | Survival | 1, 2, 3 |
| “C” | 10 | HG | Novel | Survival | 1, 2, 3 |
| “D” | 4 | Std | Novel | Non-Survival | * |

* See Tissue Heating in the Anticipated Complications section

Surgery (Groups A-C). Rats can be 10 weeks of age at the time of surgery. Although this age does not represent the Veteran population, use of younger rats reduces the risk of surgical complications during this proof-of-concept study. Younger rats are also more likely to fit into the small MRI bore. During experiments, rats can be anesthetized with isoflurane, maintained at 37° C. with a warming pad, and their vitals can be recorded. The abdomen and the ventral neck can be prepared for surgery and position the rat supine. Using established procedure, while the rat is on an electrically-grounded, pressurized, vibration-suppressing floating air table inside a Faraday cage the midline abdomen can be incised from the sternal border caudally 8 cm. The abdominal skin and muscle can be retracted, free the stomach from mesentery, and the overlying lobe(s) of liver can be retracted to expose the gastroesophageal junction. A multi-channel cuff can be placed around the distal esophagus per Table 1. For traditional cuffs, the diameter of the cuff can be secured with locking sutures positioned through anchor points on the cuff. A pair of fine wire electrodes can be implanted near the diaphragm to record diaphragm electromyograms (EMGD). The neck can be moved to where a midline incision is made. The underlying submandibular gland and sternomastoid muscle can be retracted to expose the left cVN. The nerve can be carefully dissected free from the carotid sheath and a tripolar recording cuff can be positioned around the nerve. The process can be repeated for the right cVN. At the end of the surgery, all skin incisions can be closed with simple interrupted sutures of non-absorbable monofilament and further coated with Dermabond.

Surgery (Groups B-C). For rats in Groups B and C, the surgery can be aseptic and antibiotics and analgesics can be provided prior to and after their surgery. During surgery, an interscapular space on the back for incision can be prepared. Leads from the sVNS and cVN cuffs can be routed to a percutaneous exit site on the back using our established techniques. The sVNS lead can be routed through the abdominal muscle, leaving sufficient slack to ensure that if tension develops on the lead it does not transmit to the cuff. The abdominal muscle can be sutured closed. A subcutaneous tunnel from the abdomen to the interscapular percutaneous exit site can be created while resting the rat in a lateral recumbent position. The dorsal skin can be incised to expose the underlying lead which can be pulled through the skin. This process can be repeated for the cVN leads. The rat can be positioned prone. A finger trap (Roman Sandal) suture can be placed around each externalized lead to anchor it in place using a non-absorbable monofilament per our current protocol. Following sVNS recruitment surface collection (see below), the rat can be returned to the housing facility where it can recover.

Mapping sVNS Parameters to Vagus Axon Recruitment Using Electroneurograms

sVNS Recruitment Surfaces. At the time of implant (Groups A-C) and 20 weeks after implant (Groups B-C), sVNS recruitment surfaces can be collected. To generate these surfaces, the sVNS cuff can be connected to a programmable Tucker Davis Technologies (TDT) RZ6 stimulator and cVN recording cuffs can be connected to a TDT PZ5 amplifier/digitizer and RZ2 Bioamp Processor sampling at 25 kHz. Diaphragm EMG can also be recorded at the time of implant. Initially, a supramaximal stimulus can be applied through all cathodes to recruit all sVN axons resulting in a maximal response by which to normalize all other responses. Using the described techniques, the input-output response of the stimulating cuff can be characterized by recording bilateral compound action potentials (CAPs) while sweeping sVNS from 0-3 mA and 0-1000 μs at 30 Hz. Each cathode can be tested independently. Additionally, the two cathodes that maximally recruit the ventral and dorsal sVN can be determined and a recruitment surface can be collected while delivering bilateral sVNS on both cathodes. All stimuli can be biphasic, charge-balanced. At the time of surgery, real-time analysis of the EMG_{D} can reveal if a stimulus spilled over to the phrenic nerves causing diaphragm contraction. If a stimulus is found to activate the diaphragm, no stronger stimulus need be tested. Once finished, the diaphragm electrodes can be removed.

Analysis. During post-hoc analysis, CAPs can be assigned to different groups (Aβ, Aδ, c) based on delay from stimulus onset to CAP arrival. Each CAP can be rectified (absolute value) and integrated to quantify the CAP. Each CAP within a group can then be normalized by the maximum CAP (CAPₘₐₓ) for that group so that each recorded response is interpreted on a scale of 0-100% of CAPₘₐₓ as a function of the PA, PW, and channel, thus producing normalized recruitment surfaces and fully characterizing the recruitment capabilities of the cuff. For rats in Groups B-C, the recruitment surfaces collected at surgery can be compared to those collected 20 weeks later. Using an ANOVA, it can be determined if encapsulation/time, channel number, sex, PA, PW, or diet significantly affected recruitment. If these recruitment surfaces do not differ (possibly after accounting for a shift in PA due to encapsulation), this can indicate that the novel cuff is stable. The recruitment surfaces obtained at the time of surgery can be compared. If the surfaces are similar, it can suggest that the novel cuff performed on par with the standard cuff. The maximum normalized values obtained during unilateral sVNS can be investigated. A value of 100% suggests that a single cathode recruited all axons in the underlying nerve (either ventral or dorsal sVN). Lesser values suggest that some axons were not near the active electrode and may be distributed around the esophagus. This can suggest that multi-channel stimulation or use of larger cathodes is required to achieve full activation. Understanding the number of active channels required in a cuff to fully recruit sVN axons is critical to effectively delivering optimal bilateral sVNS. Further, an observed response in both the left and right cervical recording cuffs during unilateral stimulation can suggest that axons from both sVN were near the active cathode and/or some axons crossover from one sVN to the other via a plexus.

Mapping sVNS Parameters to Brain Activation and Inhibition Using fMRI.

fMRI. Approximately 20 weeks after surgery, rats in Groups B-C can be anesthetized and the sVNS recruitment surfaces can be recollected. From these, which cathodes maximally recruit the ventral and dorsal sVN can be determined. On the next day, high-resolution fMRI during unilateral and bilateral sVNS with the chosen cathodes can be collected. The anesthetized rat can be positioned in the MRI cradle and respiration rate can be monitored throughout the experiment. An isoflurane level can be maintained as close to 1% as possible while maintaining a respiration rate near 60 breaths per minute. Prior to scanning, the sVNS percutaneous lead wires can be attached to the TDT stimulator via an MRI-compatible cable. An 11.1 T Bruker Avance III HD MR system with an in-plane resolution of 100×100 μm can be used to obtain scans. Scans can include a high-resolution anatomical scan and scans during sVNS trains using a T1-weighted Fast Spin-Echo (FSE) sequence. sVNS can be applied at 30 Hz and PA/PW combinations that capture salient features of the sVNS recruitment surface. The duty cycle can be 20 s off, 10 s on, repeating over 150 s, producing 5 presentations of each stimulus train.

Analysis. The anatomical scan can be normalized and aligned to the Waxholm brain template. FSL can be used to perform a registration between the fMRI and the anatomical data. After registration, normalization, and smoothing, a generalized linear model to can be applied these data in which stimulus “off” and “on” conditions are contrasted against each other with particular focus on the VMH, DMN, LH, VTA, and central amygdala. Multivariate statistical analyses can be conducted on these data in collaboration with the BRRC-contracted statistician to determine if the activation is affected by the presence of stimulus (on vs off), intensity of stimulus (PA, PW, PAxPW), sex of the rat, or diet. Sufficient data can be obtained to begin constructing a sVNS-specific connectome. Using these maps, how changes in sVNS parameters are represented in the fMRI data can be determined, noting differences between unilateral and bilateral sVNS. The data from unilateral ventral sVNS can be compared to data obtained with ventral sVNS through a nerve (not esophageal) cuff from an ongoing Merit Review.

### Evaluating the Effects of a Chronically-Implanted Novel Cuff on Esophageal Elasticity and sVN Health.

Esophageal Distension Pressure. The pressure required to distend the esophagus by a fixed volume can be measured. Briefly, a balloon catheter can be attached to a programmable syringe pump and a calibrated pressure transducer. The circumference around the esophagus can be measured to find its approximate diameter. Knowing the approximate diameter of the esophagus, the volume required to engorge the balloon to a diameter that is approximately 25% larger than the nominal diameter of the esophagus can be determined, representing the increase in size that can accompany swallowing of a food bolus. An incision can then be made in the greater curvature of the stomach, any stomach contents can be aspirated, and the collapsed balloon can be introduced into the distal esophagus via the gastroesophageal sphincter. The pressure required to distend (P_{D}) the balloon to the predetermined volume while filled with fluid at 1 mL/sec can be measured. Fluid can be aspirated and the trial can be repeated twice more with 5 minute breaks between trials. For rats in Group A, P_{D} can be measured at the time of implant and the stomach can already be exposed. P_{D} can be measured under two scenarios: with the cuff implanted (and “locked” to a fixed diameter via suture) and without the cuff implanted. For rats in Groups B-C, P_{D} can be measured during a final, non-survival surgery following fMRI collection and the rat can undergo the same surgical procedure detailed above to expose the stomach (Surgery (Groups A-C)). After the P_{D} is measured, the rat can be euthanized with an overdose of sodium pentobarbital.

Analysis. P_{D} can be measured under three scenarios: no implant (mimicking naïve esophagus); an implanted traditional cuff that is sutured closed (mimicking encapsulation); an implanted and encapsulated helical cuff. Using an ANOVA, it can be determined if scenario type affects P_{D}. If there is no significant difference between the naïve esophagus and that implanted with the helical cuff, it suggests that the novel cuff is mechanically safe and unlikely to promote stenosis.

Microscopy. 4 rats from Groups A, B, and C (N=12) can be randomly selected for perfusion of fixative and removal of ventral and dorsal sVN segments proximal to the cuff where no nerve damage would be expected. Similarly, sections under the cuff can be removed. Sections can be placed in fixative and transported to UF, which can process the samples for both light microscopy using a toluidine blue and transmission electron microscopy (TEM) using osmium tetroxide. The core can return the physical samples and digital images of them.

Analysis. Light microscopy images can be analyzed to determine if there was myelinated axon or gross nerve damage. Average g-ratio of myelinated axons and percentage of myelinated axons exhibiting swelling or degeneration can be quantified. TEM images can be analyzed to determine if there is a change in the density of unmyelinated axons. Within a given rat, the proximal and distal images can be compared. Images from rats in Groups B and C can be compared to rats in Group A. If there is no significant difference in these metrics, it can suggest that the novel cuff caused no nerve damage while implanted.

### Anticipated Complications and Alternative Approaches

Tissue Heating. The 11.1 T MRI may heat tissue at the electrodes. Preliminary studies showed no evidence of tissue damage due to heating, but the cuff used was different from that being designed and the field strength was weaker. Although the manufacturers do not believe heating will occur, cuffs can be implated in Group D during non-survival experiments before chronically implanting rats in Groups B or C. At the same time, fiber optics can be implanted at the sVNS and cVN recording cuffs. The fiber optics can attach to an MRI-safe fiber optic thermometer. While running the imaging sequence, internal temperatures can be monitored. This approach provides the most accurate assessment of temperature change. A temperature increase to 42-44° C. or ≥45° C. would suggest the possibility of reversible or irreversible damage, respectively. If heating to or above 42° C. is observed, it can be reduced by altering the imaging sequence, the lead routing, and/or the cuff design. If these do not solve the problem, the experiments can be conducted with the 7 T magnet.

fMRI Distortion. It is contemplated that the helical cuff or the percutaneous leads do not create distortion in our fMRI data, but there is a risk that the cVN recording cuffs can create distortion. This can be assessed early during the Tissue Heating tests prior to implanting rats in Groups B and C. If the fMRI data suggests that the cVN cuffs cause distortion, then they are not implanted permanently. Instead, the cVN cuffs can be explated at the end of the sVNS implant surgery. The recruitment surface data from the implant surgery can be used to make decisions as to what channels to use for unilateral and bilateral sVNS. At the conclusion of the study, cVN recording cuffs can be re-implanted to assess long-term changes in the recruitment surfaces

Infection. While there have been no instances of infection in 40 rats with percutaneous leads over a period of 3-5 months, infection is always a risk. To minimize the chance of infection, rats can be checked daily at their percutaneous lead site and antibiotics can be administered should any signs of infection present.

Esophageal Stenosis. Although the novel cuff can prevent stenosis, formation of fibrotic tissue that reduces esophageal elasticity could arise. If the esophagus resists expansion, it can prevent passage of food to the stomach. This has been observed in rats. If stenosis occurs, a cuff with a larger pitch can be used, which can be less electrically optimal but can increase the chances of long-term implant success.

### INNOVATION

The example is innovative because it discloses a novel cuff and assesses its safety and efficacy. It generates unilateral and bilateral sVNS data using a high-field 11.1 T MRI that can produce the highest resolution data of any sVNS study. This example demonstrates that the novel interface can be implanted chronically without the risk of stenosis and can be used in a high-field MR environment. Even if the cuff is not compatible with the 11.1 T magnet, achieving the same outcomes with the 7 T magnet is considered very successful. The cuff can serve as a powerful tool in future studies of sVNS optimization. Further, the cuff can be resized for mice, opening the door to bilateral sVNS studies in genetic knockout and transgeneic models, including optogenetic, that do not exist as rats. Further, the cuff can be used with humans when sized appropriately.

The following is claimed:

1. A cuff for providing vagus nerve stimulation to a subject having an esophagus, the cuff comprising:
a helical body that is configured to wrap around at least a portion of the esophagus of the subject, the helical body having an inner surface, the inner surface having a length and a width, wherein the inner surface defines an interior space that is configured to receive the esophagus;
a first electrode coupled to the helical body; and
at least one additional electrode coupled to the helical body that is configured to provide a polarity opposite of the first electrode.

2. The cuff of claim 1, wherein the least one additional electrode comprises a plurality of electrodes.

3. The cuff of claim 1, wherein the first electrode is a cathode, wherein the at least one additional electrode is an anode.

4. The cuff of claim 2, wherein the first electrode and the plurality of electrodes are helical.

5. The cuff of claim 1, further comprising a second electrode that is configured to provide a polarity that is the same as the first electrode, wherein the first and second electrodes are spaced along the width of the inner surface of the helical body.

6. The cuff of claim 5, wherein the at least one additional electrode is positioned between the first and second electrodes.

7. The cuff of claim 5, wherein the at least one additional electrode comprises a plurality of electrodes spaced along the length of the inner surface of the helical body.

8. The cuff of claim 7, wherein the plurality of electrodes spaced along the length of the inner surface of the helical body comprise a first row of electrodes and a second row of electrodes, wherein the first row is spaced from the second row along the width of the inner surface of the helical body.

9. The cuff of claim 5, wherein the second electrode extends a majority of the length of the inner surface of the helical body.

10. The cuff of claim 1, wherein the first electrode extends a majority of the length of the inner surface of the helical body.

11. The cuff of claim 1, wherein the helical body has a consistent pitch.

12. The cuff of claim 1, wherein the helical body has a consistent radius.

13. The cuff of claim 1, wherein the helical body is configured to resiliently expand to increase a diameter of the interior space defined by the inner surface of the helical body to permit swallowing of the subject.

14. A method of providing vagus nerve stimulation to a subject having an esophagus and a vagus nerve, the method comprising:
positioning a cuff around the esophagus of the subject, the cuff comprising:
a helical body that is configured to wrap around at least a portion of the esophagus of the subject, the helical body having an inner surface, the inner surface having a length and a width, wherein the inner surface defines an interior space that is configured to receive the esophagus;
a first electrode coupled to the helical body; and
at least one additional electrode coupled to the helical body that is configured to provide a polarity opposite of the first electrode; and
applying stimulation to the vagus nerve of the subject using the cuff.

15. The method of claim 14, wherein the stimulation is unilateral stimulation.

16. The method of claim 14, wherein the stimulation is bilateral stimulation.

17. The method of claim 14, wherein the subject is a human subject.

18. The method of claim 14, wherein the subject is an animal subject.

19. The method of claim 14, wherein applying stimulation to the vagus nerve of the subject using the cuff comprises applying stimulation to a ventral subdiaphragmatic vagus nerve of the subject.

20. The method of claim 14, wherein applying stimulation to the vagus nerve of the subject using the cuff comprises applying stimulation to a dorsal subdiaphragmatic vagus nerve of the subject.
